(19) United States
(12) Patent Application Publication
(10) Pub. No.: US 2006/0093571 A1
(43) Pub. Date: May 4, 2006

(54) Hair and skin protecting compositions based on esters or ethers of betulin

(76) Inventor: Jan Glinski, New Fairfield, CT (US)

Correspondence Address: COLEMAN SUDOL SAPONE, P.C., 714 COLORADO AVENUE, BRIDGE PORT, CT, 06605-1601, US

(21) Appl. No.: 11/262,687

(22) Filed: Oct. 31, 2005

Related U.S. Application Data
(60) Provisional application No. 60/622,983, filed on Oct. 29, 2004.

(51) Int. Cl. C07J 53/00; C07D 311/94; A61K 8/63; A61K 8/49

(52) U.S. Cl. 424/73; 552/511; 549/381

(57) ABSTRACT

Personal care compositions and formulations, which include conventional concentrations of known hair and skin altering components and a cosmetically acceptable amount of oil-soluble betulin and allo-betulin esters or ethers derived from betulin or allo-betulin in effective amounts are disclosed.

### RELATED APPLICATIONS

This application claims the benefit of priority of provisional application U.S. Ser. No. 60/622,983, filed Oct. 29, 2004, which is incorporated by reference in its entirety herein.

### FIELD OF THE INVENTION

This invention relates to personal care compositions, preferably for use on keratinous tissue, including hair and skin featuring esters or ethers of betulin and allo-betulin (“esters or ethers of betulin”), and, more particularly, to protecting, conditioning, and styling compositions advantageously having improved substantive conditioning and protection with good keratin adherence.

### DESCRIPTION OF THE PRIOR ART

Betulin and other closely related pentacyclic triterpenes such as betulinic acid, ursolic acid, and oleanolic acid are known to inhibit enzymatic activity of elastase, which is a key enzyme contributing to skin aging (Ying, Qi-Long, Rinehart, A., Simone, R., Sanford, R., Cheronis, J. C., “Inhibition of human leukocyte elastase bu ursolic acid”, Biochem. J., 277, 521-526 (1991); Glinski, J. A., Chow, G., Skoog, M., Matteo, M., “Inhibition of elastase by pentacyclic triterpenes”, 50^{th} Meeting of the American Society of Pharmacognosy, San Jose, Puerto Rico, August 1989). Plant extracts containing betulin have been demonstrated to promote skin elasticity and suppleness by its influence on collagen synthesis by the fibroblasts. Skin conditioning, anti-cellulite effect, anti-wrinkling effect and improvements in skin color were also mentioned in the literature (Matsumoto, Katsuo; Tsuruoka, Hiroki; Fujiwara, Norio; Nishimori, Yasutomo; Kenjo, Yukiko:, “Photoaging inhibitor and skin-care preparation”, U.S. Pat. No. 6,207,711 assigned to Pola Chemical Industries; Suk Ha Cho, Keith Gottlieb, Uma Santhanam, “Cosmetic compositions containing betulinic acid”, U.S. Pat. No. 5,529,769, assigned to Cheseborough Pond). They were also shown to stimulate hair growth (Dunlop D. S, Guskey S. M., Leyba, V. E., Royce, D. A., Anti-dandruff and conditioning shampoos containing polyalkylene glycols and cationic polymers, U.S. Pat. No. 6,451,300 assigned to Procter and Gamble). Enzyme inhibitory processes reflect general tendency of betulin and other pentacyclic triterpenes to bind reversibly to proteins mostly, but not exclusively, through hydrophobic interactions. Common pentacyclic triterpenes such as betulin, betulinic acid, ursolic acid, and oleanolic acid are solids characterized by high melting point and poor solubility in polar and non-polar solvents. Poor solubility of pentacyclic triterpenes is a serious obstacle in formulating these compounds into commercial applications. Proposed cosmetic and pharmaceutical applications rely on solid form, powder applications, as well as dispersions in the aqueous and oil phase (U.S. Pat. No. 5,529,769).

### SUMMARY OF THE INVENTION

In accordance with this invention, provided are various personal care compositions and formulations, which include conventional concentrations of known hair and skin altering components and a cosmetically acceptable amount of oil-soluble esters or ethers of betulin or allo-betulin in effective amounts.

More specifically, in accordance with this invention provided are the personal care composition for treatment of hair or skin comprising:

(i) a cosmetically acceptable medium comprising at least one cosmetically acceptable additive selected from surfactants (anionic, cationic, amphoteric, and nonionic) emulsifiers, oils, emollients, conditioning agents, protecting agents, fixing agents, polymers, oxidizing agents, reducing agents, dyes, uv absorbers, coloring agents, diluents, solvents, preservatives and

(ii) oil-soluble ester or ether of betulin or allo-betulin comprising:
(a) Betulin or allo-betulin moiety
(b) Ester moiety or ether moeity; and

(iii) optionally, preservatives, anti-oxidants.

The compositions are designed for treatment of hair and skin according to usual treatment procedures.

### DETAILED DESCRIPTION OF THE INVENTION

The following terms are used to describe the present invention. Where a term is not defined, the meaning which is used is that which one of ordinary skill would assign to the term within the context of its use in the present invention.

The term “effective amount” is used throughout the specification to describe concentrations or amounts of compounds or components which are effective within the context of their use in the present invention.

The term “personal care composition” or “personal care product” is used throughout the specification to describe a cosmetic or toiletry product which is preferably used on or in contact with the hair, skin and/or nails and which include effective concentrations of one or more of the esters or ethers of betulin according to the present invention. Personal care products include, for example, cosmetics, floating bath oils, after shaves, creams, lotions, deodorants, including stick deodorants, pre-electric shave lotions, after-shave lotions, antiperspirants, deodorants, shampoos, conditioners and rinses and related products, among others, including skin care products such as skin lotions and moisturizers, eye makeups, body shampoos, protective skin formulations, lipsticks, lip glosses, after-bath splashes, presun and sun products, including sunscreens, among numberous other products. Virtually any cosmetic product of a chemical or biochemical nature which comes into contact with the hair or skin and which may include effective amounts or concentrations of one or more of the compositions according to the present invention may be considered a personal care product/composition according to the present invention.

The term “acyl” is used to describe a C(O)R group, preferably a C₂ to C₃₂ acyl group, more preferably a C₂ to C₂₄ acyl group, where R is a hydrocarbyl group (optionally containing one or more unsaturated bonds) preferably containing from one to 31 carbon atoms, and preferably containing one or more double bonds. Acyl groups according to the invention may be substituted with one or more substituent groups (preferably, within context, up to six substituents) as otherwise described herein.

The term “ether” is used to describe an OR group, preferably a C₁ to C₃₂ OR group, where R is a hydrocarbyl group (optionally containing one or more unsaturated bonds) preferably containing from one to 32 carbon atoms and more preferably containing one or more double bonds. Ether groups according to the invention may be substituted with one or more substituent groups (preferably, within context, up to six substituents) as otherwise described herein.

The term “alkyl” is used to describe a monovalent fully saturated hydrocarbon, which when optionally and preferably containing at least one double bond (e.g., alkene, alkadiene, etc.) more preferably containing more than one double bond, may instill antioxidant properties to a final personal care product according to the present invention.

Betulin is a substance of a resinous nature, obtained from the outer bark of the common European birch (Betula alba)—called also birch camphor. Compounds which are used in the present invention include esters or ethers of betulin as otherwise described herein.

The term “betulin esters or ethers” or “allo-betulin esters or ethers” (collectively, for purposes of convenience in describing the present invention “esters or ethers of betulin”) are esters or ethers of betulin or allo-betulin as otherwise described hereinbelow. Betulin esters or ethers or allo-betulin esters or ethers employed in the compositions of the invention preferably comprise structures of the formulas given below:

Betulin Esters or Ethers 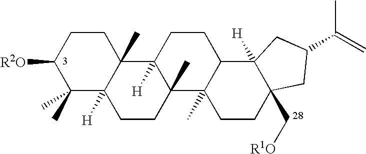
where R¹ and R² are independently H, an optionally substituted acyl group (preferably a C₂-C₃₂ acyl group, more preferably a C₂-C₂₄ acyl group, or, forms an optionally substituted ether group with the oxygen to which it is attached, and in the case of an ether group, is preferably a C₁-C₃₂ hydrocarbyl group, more preferably a C₁-C₂₄ hydrocarbyl group with the proviso that at least one of R¹ and R² is an acyl or a hydrocarboyl group (ether), wherein said optionally substituted acyl group or ether group is linear or branch-chained and preferably contains one or more double bonds.

Allobetulin Esters or Ethers 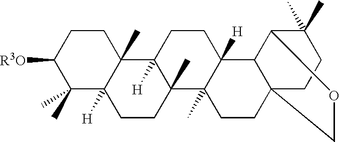
where R³ is an optionally substituted acyl group or forms an optionally substituted ether group with the oxygen to which it is attached (preferably, a C₂-C₃₂ acyl group, more preferably a C₂-C₂₄ acyl group, or, in the case of an ether group, is preferably a C₁-C₃₂ hydrocarbyl group, more preferably a C₁-C₂₄ hydrocarbyl group), wherein said optionally substituted acyl group or ether group is linear or branch-chained and preferably contains one or more double bonds.

Esters or ethers of betulin are in a liquid, semisolid, or solid state depending on the fatty chain substituent. Betulin oleate, which contains an unsaturated alkyl chain, is a thick liquid, while betulin stearate, based on saturated C₁₈ alkyl residue is in a semi-solid. Esters or ethers of betulin are essentially oil-soluble and form a clear, humidity resistant, hydrophobic film when cast upon a support surface. Further, esters containing a minimum of two conjugated double bonds display antioxidant properties. In the cosmetic compositions of the present invention, a wide variety of enhanced skin and hair altering effects are achieved by the use of cosmetically acceptable compositions comprising between about 0.01 to 50% by weight, preferably about 0.1 to about 20% by weight, preferably about 0.01 to about 10% by weight of the final personal care product based on the total weight of the composition of the esters or ethers of betulin to a standard or equivalent hair or skin formulation.

The term “hydrocarbyl group” refers to an optionally substituted group containing carbons and hydrogens of various size and being linear or branch-chained, the hydrocarbyl group being an alkyl group (no unsaturation) or with one or more unsaturated bonds (alkene or alkyne), but preferably containing one or more double bonds.

The term “optionally substituted” refers to an acyl group or ether group, which, is optionally substituted with hydroxyl (OH), halogen (F, Cl, Br, I), (C₁-C₁₂)(preferably, C₁-C₆) alkoxy, epoxy, C₁-C₆ carboxy, or an amine group which is optionally substituted with one or two optionally substituted C₁-C₆ alkyl groups.

Compositions according to the present invention preferably include an effective amount of a conditioning agent. Any known conditioning agent is useful in the compositions of this invention. Conditioning agents function to improve the cosmetic properties of the hair, particularly softness, untangling, feel, and static electricity and may be in liquid, semi-solid, or solid form such as oils, waxes, or gums.

Conditioning agents may be chosen from synthetis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds. Preferred conditioning agents include cationic polymers and silicones.

The term “oil” is used throughout the specification to describe any of various lubricious, hydrophobic and combustible substances obtained from animal, vegetable and mineral matter. Oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Non-polar” oils are generally oils such as petrolatium or mineral oil or its derivatives which are hydrocarbons and are more hydrophobic and lipophilic compared to synthetic oils, such as esters, which may be referred to as “polar” oils. It is understood that within the class of oils, that the use of the terms “non-polar” and “polar” are relative within this very hydrophobic and lipophilic class, and all of the oils tend to be much more hydrophobic and lipophilic than the water phase which is used in the present invention.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmacuetical products. Cosmetic grade oils are preferred oils for use in the present invention.

Additional oils for use in the present invention may include, for example, mono-, di- and tri-glycerides which may be natural or synthetic (derived from esterification of glycerol and at least one organic acid, saturated or unsaturated, such as for example, such as butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂ to C₃₀ (or higher) fatty esters (other than the glyceride esters, which are described above) or any other acceptable cosmetic emollient.

The synthetic oils include polyolefins, e.g., poly-α-olefins such as polybutenes, polyisobutenes and polydecenes. The polyolefins can be hydrogenated.

The mineral oils suitable for use in the compositions of the invention include hexadecane and paraffin oil.

Additional suitable animal and vegetable oils include sunflower, olive, corn, soy, avocado, jojoba, squash, raisin seed, sesame seed, walnut oils, fish oils, glycerol tricaprocaprylate, Purcellin oil or liquid jojoba.

Additional suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot.

Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax.

The cationic polymers (other than the poly (alkyl) vinyllactam polymers according to the invention) that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a molecular weight the average number of which falls between about 500 and 5,000,000 and preferably between 1000 and 3,000,000.

The preferred cationic polymers are chosen from among those containing units including primary, secondary, tertiary, and/or quaternary amine groups that may either form part of the main polymer chain or a side chain.

Useful cationic polymers include known polyamine, polyaminoamide, and quaternary polyammonium types of polymers, such as:

(1) homopolymers and copolymers derived from acrylic or methacrylic esters or amides. The copolymers can contain one or more units derived from acrylamides, methacrylamides, diacetone acrylamides, acrylamides and methacrylamides, acrylic or methacrylic acids or their esters, vinyllactams such as vinyl pyrrolidone or vinyl caprolactam, and vinyl esters. Specific examples include: copolymers of acrylamide and dimethyl amino ethyl methacrylate quaternized with dimethyl sulfate or with dimethyl halogenide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name GAFQUAT by International Specialty Products; the dimethyl amino ethyl methacrylate/vinyl caprolactam/vinyl pyrrolidone terpolymers, such as the product sold under the name GAFFIX VC 713 by International Specialty Products; the vinyl pyrrolidone/methacrylamidopropyl dimethylamine copolymer, marketed under the name STYLEZE CC 10 by International Specialty Products; and the vinyl pyrrolidone/quaternized dimethyl amino propyl methacrylamide copolymers such as the product sold under the name GAFQUAT HS 100 by International Specialty Products.

(2) derivatives of cellulose ethers containing quaternary ammonium groups, such as hydroxy ethyl cellulose quaternary ammonium that has reacted with an epoxide substituted by a trimethyl ammonium group.

(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.

(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.

(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.

(6) water-soluble polyamino amides prepared by polycondensation of an acid compound with a polyamine. These polyamino amides may be reticulated.

(7) derivatives of polyamino amides resulting from the condensation of polyalcoylene polyamines with polycarboxylic acids followed by alcoylation by bi-functional agents.

(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.

(9) the cyclopolymers of alkyl dialyl amine or dialkyl diallyl ammonium such as the homopolymer of dimethyl diallyl ammonium chloride and copolymers of diallyl dimethyl ammonium chloride and acrylamide.

(10) quaternary diammonium polymers such as hexadimethrine chloride. Polymers of this type are described particularly in U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

(11) quaternary polyammonium polymers, including, for example, Mirapol® A 15, Mirapol® AD1, Mirapol® AZ1, and Mirapol® 175 products sold by Miranol.

(12) the quaternary polymers of vinyl pyrrolidone and vinyl imidazole such as the products sold under the names Luviquat® FC 905, FC 550, and FC 370 by BASF.

(13) quaternary polyamines.

(14) reticulated polymers known in the art.

Other cationic polymers that may be used within the context of the invention are cationic proteins or hydrolyzed cationic proteins, polyalkyleneimines such as polyethyleneimines, polymers containing vinyl pyridine or vinyl pyridinium units, condensates of polyamines and epichlorhydrins, quaternary polyureylenes, and derivatives of chitine.

Preferred cationic polymers are derivatives of quaternary cellulose ethers, the homopolymers and copolymers of dimethyl diallyl ammonium chloride, quaternary polymers of vinyl pyrrolidone and vinyl imidazole, and mixtures thereof.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and mixtures thereof.

Suitable polyalkyl siloxanes include polydimethyl siloxanes with terminal trimethyl silyl groups or terminal dimethyl silanol groups (dimethiconol) and polyalkyl (C₁-C₂₀) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes preferably having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers.

The silicones may be used in the form of emulsions, nano-emulsions, or micro-emulsions.

The conditioning agent can be a protein or hydrolyzed cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C₁-C₁₈ alkyl.

Hydrolyzed proteins include Croquat L, in which the quaternary ammonium groups include a C₁₂ alkyl group, Croquat M, in which the quaternary ammonium groups include C₁₀-C₁₈ alkyl groups, Croquat S in which the quaternary ammonium groups include a C₁₈ alkyl group and Crotein Q in which the quaternary ammonium groups include at least one C₁-C₁₈ alkyl group. These products are sold by Croda.

The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

According to the invention, the conditioning agent can be a ceramide type of compound such as a ceramide, a glycoceramide, a pseudoceramide, or a neoceramide. These compounds can be natural or synthetic. Compounds of the ceramide type are, for example, described in Patents pending DE4424530, DE4424533, DE4402929, DE4420736, WO95/23807, WO94/07844, EP-A-0646572, WO95/16665, FR-2 673 179, EP-A-0227994, WO 94/07844, WO 94/24097, and WO 94/10131. Ceramide type compounds useful herein include 2-N-linoleoyl amino-octadecane-1,3-diol, 2-N-oleoyl amino-octadecane-1,3-diol, 2-N-palmitoyl amino-octadecane-1,3-diol, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl) malonamide, N(2-hydroxy ethyl)-N-(3-cetoxyl-2-hydroxy propyl) amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide.

The conditioning agent can be any fatty amine known to be useful as a conditioning agent.

The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic ester acids including mono-, di-, tri- and tetra-carboxylic acids.

The conditioning agent can be a fluoridated or perfluoridated oil. Fluoridated oils include perfluoropolyethers described in EP-A-486135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

Of course, mixtures of two or more conditioning agents can be used.

The conditioning agent or agents can be present in an amount of 0.001% to 20%, preferably from 0.01% to 10%, and even more preferably from 0.1% to 3% by weight based on the total weight of the final composition. In compositions which are emulsions (oil-in-water or water-in-oil) used to produce final products, the amount of conditioning agent in the form of an oil or other component as described herein above may range from as little as 5% to more than 95% by weight.

The composition of the invention can contain one or more protecting agents to prevent or limit the degrading effects of natural physical and/or chemical assaults on the keratinous materials. Personal care products using these components may be useful as skin protective agents or sun-blocking compositions as creams or lotions, although the inclusion of these components in any personal care product exposed to the sun may also be useful.

The protecting agent can be chosen from hydrosoluble, liposoluble and water-insoluble UV filters, antiradical agents, antioxidants, vitamins and pro-vitamins.

Organic UV absorbers or filters (systems that filter out UV rays) can be chosen from among hydrosoluble or liposoluble filters, whether siliconated or nonsiliconated, and mineral oxide particles, the surface of which may be treated.

Hydrosoluble organic UV filters may be chosen from para-amino benzoic acid and its salts, anthranilic acid and its salts, salicylic acid and its salts, hydroxy cinnamic acid and its salts, sulfonic derivatives of benzothiazoles, benzimidizoles, benzoxazoles and their salts, sulfonic derivatives of benzophenone and their salts, sulfonic derivatives of benzylidene camphor and their salts, derivatives of benzylidene camphor substituted by a quaternary amine and their salts, derivatives of phthalydene-camphosulfonic acids and their salts, sulfonic derivatives of benzotriazole, and mixtures thereof.

Hydrophilic polymers which have light-protective qualities against UV rays can be used. These include polymers containing benzylidene camphor and/or benzotriazole groups, [substituted by quaternary sulfonic or ammonium groups].

Suitable liposoluble organic UV filters include derivatives of para-aminobenzoic acid, such as the esters or amides of para-aminobenzoic acid; derivatives of salicylic acid; derivatives of benzophenone; derivatives of dibenzoyl methane; derivatives of diphenyl acrylates; derivatives of benzofurans; UV filter polymers containing one or more silico-organic residues; esters of cinnamic acid; derivatives of camphor; derivatives of trianilino-s-triazine; the ethylic ester urocanic acid; benzotriazoles; derivatives of hydroxy phenyl triazine; bis-resorcinol-dialkyl amino triazine; and mixtures thereof.

The liposoluble (or lipophilic) organic UV filter according to the invention can be chosen from octyl salicylate; 4-tert-butyl-4′-methoxy dibenzoyl methane; octocrylene; 4-methoxy cinnamate; 2-ethylhexyl [2-ethylhexyl 4-methoxycinnamate]; and 2-(2H-benzotriazol-2-yl)-4-methyl-6-[2-methyl-3-[1,3,3,3-tetramethyl-1-[(trimethyl silyl)oxy] disiloxanyl]propynyl]phenol.

Other UV filters particularly preferred for use herein are derivatives of benzophenones such as 2-hydroxy-4-methoxy benzophenone-5-sulfonic acid, 2-hydroxy-4-methoxy benzophenone, derivatives of benzalmalonates such as poly dimethyl/methyl (3(4-(2,2-bis-ethoxy carbonyl vinyl)-phenoxy)-propenyl) siloxane, derivatives of benzylidene camphor such as b-b′camphosulfonic [1-4 divinylbenzene] acid and derivatives of benzimidazole such as 2-phenyl-benzimidazol-5-sulfonic acid.

Water-insoluble UV filters include various mineral oxides. The mineral oxides may be selected from among titanium oxides, zinc oxides, and cerium oxides. The mineral oxides can be used in the form of ultrafine nanoparticles.

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent may be present in an amount ranging from about 0.001% to 20% by weight, preferably from about 0.01% to 10% by weight, and more preferably about 0.1 to 5% by weight of the total weight of the final composition.

The composition of the invention can contain a fixing agent. The fixing agent can be an anionic polymer chosen from polymers containing carboxylic units derived from unsaturated carboxylic mono- or diacids of the formula III:

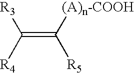

in which n is a whole number from 0 to 10, A denotes a methylene group, optionally bonded to the carbon atom of the unsaturated group or to a neighboring methylene group when n is greater than 1 by means of a heteroatom like oxygen or sulfur, R₃ denotes a hydrogen atom, a phenyl or benzyl group, R₄ denotes a hydrogen atom, a lower alkyl or carboxyl group, R₅ denotes a hydrogen atom, a lower alkyl group, a —CH₂—COOH, phenyl or benzyl group and polymers containing units derived from sulfonic acid like vinylsulfonic, styrenesulfonic, acrylamidoalkylsulfonic units.

The fixing agent can be an amphoteric polymer chosen from the polymer containing recurring units derived from:

a) at least one monomer chosen from acrylamides or methacrylamides substituted on the nitrogen with an alkyl radical,

b) at least one acid copolymer containing one or more reactive carboxyl groups, and

c) at least one basic co-monomer, such as esters with primary, secondary, tertiary, and quaternary amino substituents of acrylic and methacrylic acids and the product of quaternization of dimethylaminoethyl methacrylate with dimethyl or diethyl sulfate.

The fixing agent can be a nonionic polymer chosen from polyalkyloxazolines; vinyl acetate homopolymers; vinyl acetate and acrylic ester copolymers; vinyl acetate and ethylene copolymers; vinyl acetate and maleic ester copolymers; polyethylene and maleic anhydride copolymers; homopolymers of alkyl acrylates; homopolymers of alkyl methacrylates; copolymers of acrylic esters; copolymers of alkyl acrylates and alkyl methacrylates; copolymers of acrylonitrile and a nonionic monomer chosen from among butadiene and alkyl (meth)acrylates; copolymers of alkyl acrylate and urethane; and polyamides.

The fixing polymer can be a functionalized or unfunctionalized, silicone or non-silicone polyurethane.

The fixing polymer can be a polymer of the grafted silicone type containing a polysiloxane portion and a portion consisting of a nonsilicone organic chain, with one of the two portions forming the main chain of the polymer, and with the other being grafted onto said main chain.

The fixing agent may be present in the composition in a relative weight concentration between 0.1 and 10%, preferably 0.5 and 5%.

The composition of the invention can contain an oxidizing agent. The oxidizing agent can be chosen from the group of hydrogen peroxide, urea peroxide, alkali metal bromates, ferricyanides, persalts, and redox enzymes, optionally with their respective donor or cofactor. In a particularly preferred embodiment, the oxidizing agent is hydrogen peroxide. The oxidizing agent can be a solution of oxygenated water whose titer varies from 1 to 40 volumes.

The composition of the invention can contain at least one reducing agent in amounts from 0.01 to 30 wt %, preferably 0.05 to 20 wt % of the total weight of the composition. The reducing agents useful in the practice of this invention can be selected from thiols, like cysteine, thioglycolic acid, thiolactic acid, their salts and esters, cysteamine, and its salts or sulfites. In the case of compositions intended for bleaching, ascorbic acid, its salts and its esters, erythorbic acid, its salts and its esters, and sulfinates, like sodium hydroxymethanesulfinate can be used.

The composition of the invention can contain a dye selected from the group consisting of neutral acid or cationic nitrobenzene dyes, neutral acid or cationic azo dyes, quinone dyes, neutral, acid or cationic anthraquinone dyes, azine dyes, triarylmethane dyes, indoamine dyes and natural dyes. The dye or dyes can be present in a concentration from 0.001 to 20% and preferably 0.005 to 10 wt % based on the total weight of the composition. Coloring agents and pigments may also be included in compositions according to the present invention in similar amounts as the dyes, described above.

The composition of the invention can contain at least one amphoteric polymer or a cationic polymer selected from those commonly employed in cosmetic products such as polyquaternium-11, polyquaternium-10, polyquaternium-7, etc. Suitable amphoteric polymers include a copolymer containing at least one acrylic acid and an ammonium salt as a monomer, a monomer containing carboxy betaine, sulphobetaine, or amine oxide functionalities. The cationic or amphoteric polymer or polymers can be present in an amount of 0.01 to 10%, preferably 0.05 to 5%, and more preferably 0.1 to 3% by weight of the total weight of the composition.

In addition, the compositions according to the invention advantageously include at least one surfactant, which can be present in an amount of 0.1% and 60% preferably 1% and 40%, and more preferably 5% and 30% by weight based on the total weight of the composition. The surfactant may be chosen from among anionic, amphoteric, or non-ionic surfactants, or mixtures of them known to be useful in personal care suitable

The composition of the invention can contain one or more additional cosmetically acceptable additives chosen from conditioning agents, protecting agents, such as, for example, hydrosoluble, liposoluble and water-insoluble UV filters, antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, preservatives, hydroxy acids, cationic and nonionic polyether associative polyurethanes, polymers other than the cationic polymer described herein, vegetable oils, mineral oils, synthetic oils, polyols such as glycols and glycerol, silicones, aliphatic alcohols, colorants, bleaching agents, highlighting agents and sequestrants. These additives are present in the composition according to the invention in proportions that may range from 0 to 20% by weight in relation to the total weight of the composition. The precise amount of each additive may be easily determined by an expert in the field according to the additive's nature and its function.

The compositions according to the invention may be used to wash and treat keratinous material such as hair, skin, eyelashes, eyebrows, fingernails, lips, and hairy skin.

The compositions according to the invention can be detergent compositions such as shampoos, bath gels, and bubble baths. In this mode, the compositions will comprise a generally aqueous washing base. The surfactant or surfactants that form the washing base may be chosen alone or in blends, from known anionic, amphoteric, or non-ionic surfactants. The quantity and quality of the washing base must be sufficient to impart a satisfactory foaming and/or detergent value to the final composition. The washing base can be from 4% to 50% by weight, preferably from 6% to 35% by weight, and even more preferentially from 8% to 25% by weight of the total weight of the final composition.

The pH of the composition applied to the keratinous material is generally between 2 and 12. It is preferably between 3 and 8, and may be adjusted to the desired value by means of acidifying or alkalinizing agents that are well-known in the state of the art in compositions applied to keratinous materials. Thus, the composition of the invention can contain at least one alkalizing or acidifying agent in amounts from 0.01 to 30 wt % of the total weight of the composition.

The alkalizing agent can be chosen from ammonia, alkali carbonates, alkanolamines, like mono-, di- and triethanolamines, as well as their derivatives, hydroxyalkylamines and ethoxylated and/or propoxylated ethylenediamines, sodium or potassium hydroxide.

The acidifying agent can be chosen from mineral or organic acids, like hydrochloric acid, orthophosphoric acid, carboxylic acids like tartaric acid, citric acid, or lactic acid, or sulfonic acids and the like.

The physiological and cosmetically acceptable medium may consist exclusively of water, a cosmetically acceptable solvent, or a blend of water and a cosmetically acceptable solvent, such as a lower alcohol composed of C₁ to C₄, such as ethanol, isopropanol, t-butanol, n-butanol, alkylene glycols such as propylene glycol, and glycol ethers. However, the compositions of the invention can be anhydrous. In this case oils or emollients, such as, for example, isocetyl alcohol or octyl palmitate, can be employed as a solvent.

Generally the present cosmetic compositions are prepared by simple mixing procedures well known in the art.

The invention also has as its object a process for treating keratinous material such as the skin or hair, characterized in that it consists of applying to the keratinous materials a cosmetic composition as described above, and then eventually rinsing it with water. In the case of leave-in treatments, rinsing with water can be skipped.

So, the process according to the invention makes it possible to maintain the hairstyle, treatment, care, washing, or make-up removal of the skin, the hair, and any other keratinous material.

The compositions according to the invention may also take the form of after-shampoo compositions, to be rinsed off or not, for permanents, straightening, waving, dying, or bleaching, or the form of rinse-off compositions to be applied before or after dyeing, bleaching, perming, straightening or even between the two stages of a permanent or straightening process.

The compositions of the invention may also take the form of skin-washing compositions, and particularly in the form of solutions or gels for the bath or shower, or of make-up removal products.

The compositions of the invention may also be in the form of aqueous or hydro-alcoholic solutions for skin and/or hair care.

In general, betulin esters or ethers and/or allo-betulin esters or ether (esters or ethers of betulin) according to the present invention are included in end-use formulations in amounts ranging from about 0.01% to about 50% by weight, more preferably about 0.1% to about 20% by weight or about 0.1% to about 10% by weight, depending upon the end-use.

For example, in shampoos, rinses, conditioners, hair straighteners, hair colorants and permanent wave formulations, the ester compounds according to the present invention preferably comprise about 0.1% to about 25% by weight, more preferably about 0.25% to about 10% by weight of the final end-use hair-care composition. Other components which may be included in hair-care formulations include, for example, a solvent or diluent such as water and/or alcohol, surfactants, thickeners, coloring agents, preservatives, additional conditioning agents and humectants, among numerous others.

In the case of shave creams and gels, after-shave lotions and shave-conditioning compositions (for example, pre-electric shave formulations), the compositions according to the present invention are included in amounts ranging from about 0.1% to about 15% or more by weight, more preferably about 0.1% to about 10% by weight. Other components which may be included in these end-use compositions include, for example, water, and at least one or more of emollients, humectants and emulsifiers and optionally, other conditioning agents, medicaments, fragrances and preservatives.

In the case of skin lotions and creams, the present compositions are included in amounts ranging from about 0.1% to about 25% by weight, more preferably, about 0.25 to about 10% by weight. Additional components which may be employed in these compositions include, for example, water, emollients and emulsifers and optionally, other conditionoing agents medicaments, fragrances and preservatives.

In the case of sunscreens and skin-protective compositions, the present compositions are included in amounts ranging from about 0.01% to about 20% or more by weight, preferably about 0.1% to about 10% by weight of the final formulations. Additional components which may be employed in these compositions may include, for example, a UV absorbing composition such as para-amino benzoic acid (PABA) or a related UV absorber or a pigment such as TiO₂, water or oil, and optional components as described hereinabove, including, for example, one or more of an oil, water, suspending agent, other conditioning agents and emollients, among others.

In the case of bar and liquid soaps, compositions according to the present invention are included in amounts ranging from about 0.1% to about 10% by weight or more, preferably about 0.5% to about 10% by weight. Additional components which may be included in bar and liquid soaps include water and surfactants and optionally, bacteriacides, fragrances and colorants, among others.

Personal care product compositions according to the present invention may be presented as homogeneous compositions which compositions may include oil-in-water and water-in-oil emulsions. These emulsions generally include amounts of water, an oil or other hydrophobic component and a surfactant in amounts effective to produce an emulsion. The amount of water and oil in the emulsions may vary widely (from about 5% to about 95% by weight) and the amount of surfactant or emulsifier (to compatabilize the oil and water into an emulsion) is an amount effective to produce the emulsion, generally about 0.1% to about 20%, preferably about 1% to about 15% by weight of the emulsion. The ester compounds of the present invention may be included in the emulsiosn in amounts ranging from about 0.01% to about 50% (in certain cases as a portion of the hydrophobic component of the emulsion), preferably about 0.5% to about 25% of the ester compound is used. Any number of additional components may be added to the emulsion compositions to produce a final personal care composition. The emulsions may be used directly as lotions or creams or as a formulation base to produce final personal care products with the addition of other components, depending on the end use of the composition.

The following compositions are representative of formulations according to the present invention.

### EXAMPLE 1

### Preparation of Betulin Oleate by Direct Esterification of Betulin.

One hundred grams of betulin was combined with 75 g of oleic acid (stoichiometric amounts for mono-substitution) and 1.0 g of Vitamin E in a flask purged continuously with nitrogen. The mixture was heated on an oil bath with stirring to 190°-200° C. for 2.5-3.5 hours. The end-point of the reaction was determined either by an HPLC or TLC analysis. The content of the flask was cooled down and the final product was removed and placed in storage containers.

### EXAMPLE 2

### Preparation of Betulin Dioleate by Direct Esterfication of Betulin.

One hundred grams of betulin was combined with 150 g of oleic acid (stoichiometric amounts for di-substitution) and 1.0 g of Vitamin E in a flask purged continuously with nitrogen. The mixture was heated on an oil bath with stirring to 190°-200° C. for 3.5-5.5 hours. The end-point of the reaction was determined either by an HPLC or TLC analysis. The content of the flask was cooled down and the final product was removed and placed in storage containers.

### EXAMPLE 3

### Preparation of Betulin Stearate by Direct Esterfication of Betulin.

One hundred grams of betulin was combined with 75 g of stearic acid (stoichiometric amounts for mono-substitution) and 1.0 g of Vitamin E in a flask purged continuously with nitrogen. The mixture was heated on an oil bath with stirring to 190°-200° C. for 3.5-5.5 hours. The end-point of the reaction was determined either by an HPLC or TLC analysis. The content of the flask was cooled down and the final product was removed and placed in storage containers.

### EXAMPLE 4

### Preparation of Betulin Distearate by Direct Esterfication of Betulin.

One hundred grams of betulin was combined with 150 g of stearic acid (stoichiometric amounts for di-substitution) and 1.0 g of Vitamin E in a flask purged continuously with nitrogen. The mixture was heated on an oil bath with stirring to 190°-200° C. for 3.5-5.5 hours. The end-point of the reaction was determined either by an HPLC or TLC analysis. The content of the flask was cooled down and the final product was removed and placed in storage containers.

### EXAMPLE 5

### Preparation of Betulin Linoleate by Direct Esterfication of Betulin.

One hundred grams of betulin was combined with 75 g of linoleic acid (stoichiometric amounts for mono-substitution) and 1.0 g of Vitamin E in a flask purged continuously with nitrogen. The mixture was heated on an oil bath with stirring to 190°-200° C. for 2.5-3.5 hours. The end-point of the reaction was determined either by an HPLC or TLC analysis. The content of the flask was cooled down and the final product was removed and placed in storage containers under nitrogen.

### EXAMPLE 6

Preparation of Betulin Dilinoleate by Esterfication of Betulin.

One hundred grams of betulin was combined with 150 g of linoleic acid (stoichiometric amounts for di-substitution) and 1.0 g of Vitamin E in a flask purged continuously with nitrogen. The mixture was heated on an oil bath with stirring to 190°-200° C. for 3.5-5.5 hours. The end-point of the reaction was determined either by an HPLC or TLC analysis The content of the flask was cooled down and the final product was removed and placed in storage containers under nitrogen.

### EXAMPLE 7

### Preparation of Betulin Linolenoate by Esterfication of Betulin.

One hundred grams of betulin was combined with 75 g of linolenic acid (stoichiometric amounts for mono-substitution) and 1.0 g of Vitamin E in a flask purged continuously with nitrogen. The mixture was heated on an oil bath with stirring to 190°-200° C. for 2.5-3.5 hours. The end-point of the reaction was either by an HPLC or TLC analysis. The content of the flask was cooled down and the final product was removed and placed in storage containers under nitrogen.

### EXAMPLE 8

### Preparation of Betulin Dilinolenoate by Esterfication of Betulin

One hundred grams of betulin was combined with 150 g of linolenic acid (stoichiometric amounts for di-substitution) and 1.0 g of Vitamin E in a flask purged continuously with nitrogen. The mixture was heated on an oil bath with stirring to 190°-200° C. for 3.5-5.5 hours. The end-point of the reaction was determined either by an HPLC or TLC analysis. The content of the flask was cooled down and the final product was removed and placed in storage containers under nitrogen.

### EXAMPLE 9

Preparation of Betulin Oleate by Transesterfication of Methyl Oleate

One hundred grams of betulin was combined with 75 g of methyl oleate (stoichiometric amounts for mono-substitution) and 1.0 g of Vitamin E in a flask purged continuously with nitrogen. The mixture was heated on an oil bath with stirring to 190°-200° C. for 2.5-3.5 hours. The end-point of the reaction was determined either by an HPLC or TLC analysis. The content of the flask was cooled down and the final product was removed and placed in storage containers under nitrogen.

### EXAMPLE 10

### Conditioning Cream Rinse Formulation

| Raw material | % w/w |
|---|---|
| Phase A | |
| NaEDTA | 0.1 |
| DI water | 95.3 |
| Diazolidinyl urea/IPBC (Germall Plus, ISP) | 0.1 |
| Phase B | |
| Cetearyl alcohol (and) Behenyl trimethyl ammonium | 4 |
| chloride (Icroquat Behenyl TMS) | |
| Betulin monoleate | 0.5 |

Instructions

Heat Part A to 60° C. with moderately slow stirring. Melt Part B and add slowly to Part A with stirring until the mixture appears well mixed and homogeneous. Continue slow stirring and allow solution to cool to an ambient temperature.

### EXAMPLE 11

### Hair Moisturizer

| Raw material | % w/w |
|---|---|
| Phase A | |
| Isoeicosane | 28.00 |
| Lanolin | 7.00 |
| Beeswax | 5.00 |
| Betulin oleate | 2.00 |
| Petrolatum | 3.00 |
| Sorbitan oleate | 2.00 |
| Octyl methoxycinnamete | 2.50 |
| Propylparaben | 0.15 |
| Phase B | |
| Deionized water | 48.15 |
| Sodium borate | 0.90 |
| Propylene glycol | 0.75 |
| Methylparaben | 0.30 |
| Panthenol | 0.25 |

Add A and heat to 75° C. while stirring. Separately, combine Phase B, heat it to 75° C. Add B to A using rapid agitation and then cool to room temperature.

### EXAMPLE 12

### Conditioning Shampoo Formulation

| Raw material | % w/w |
|---|---|
| Phase A | |
| Ammonium lauryl sulfate (Standapol A, Henkel Corporation) | 15 |
| Sodium laurl sulfate (Rhodapon SB-8208/S, Rhone Poulenc) | 15 |
| Cocamidopropyl betaine (Mitratine CB, Rhone Poulenc) | 8 |
| Deionized water | 58.3 |
| Polyquaternium-11 (20%) (Gafquat 755N, ISP) | 5 |
| Phase B | |
| Lauramide DEA (Monamid 716, Mona Industries) | 2 |
| Betulin oleate (prepared according to Example 1 | 0.5 |
| Part C | |
| Diazolidinyl urea/IPBC (Germall Plus, ISP) | 0.2 |

Instructions

Heat Part A to 60° C. with moderately slow stirring for approximately ½ hr. or until solution becomes transparent. At the same time, heat Part B to 55° C. while stirring until homogeneous solution is obtained. Add Part B to Part A while continuously stirring. Remove temperature source. Once the resulting solution has reached 45° C., add Part C. Continue to stir (slowly) until the target solution has cooled to an ambient temperature.

In the conditioners and shampoo formulations, tested under the actual use conditions in comparison with similar formulations with known polymers and surfactants, the esters or ethers of betulin of the invention have excellent wet combing, excellent dry feel and softness, and excellent wet feel.

Furthermore they are characterized by excellent styling properties in terms of stiffness, humidity resistance and dry and wet feel.

### EXAMPLE 13

### Hair Relaxer

| Raw material | % w/w |
|---|---|
| Phase A | |
| Deionized water | 54.0 |
| Propylene glycol | 2.00 |
| Phase B | |
| Emulsifying wax (polawax) | 15.0 |
| Petrolatum | 8.00 |
| Hydrogenated polyisobutene | 10.0 |
| Betulin oleate | 2.00 |
| Phase C | |
| Sodium Hydroxide, 25% soln. | 8.00 |
| Phase D | |
| Propylene glycol (and) diazolidinyl urea | 1.00 |
| (and) methylparaben (and) propylparaben | |

Heat premixed A and B separately to 75° C. and add B to A with rapid mixing. Cool to 40° C. before adding C and D.

### EXAMPLE 14

### Nonaerosol Styling Spray

| Raw material | % w/w |
|---|---|
| Isodecane | 44.0 |
| SD alcohol 40 | 24.90 |
| Octyldodecyl stearoyl stearate | 18.0 |
| Isocicosane | 9.00 |
| Isostearyl hydrolyzed protein | 1.00 |
| Vitamin A palmitate | 0.10 |
| Betulin linoleate | 1.00 |
| Lanolin oil | 2.00 |

Combine ingredients and mix until uniform

### EXAMPLE 15

### Hair Bleach

The following bleaching composition was prepared (amounts expressed in grams):

Part A

| | |
|---|---|
| Potassium persulfate | 35 |
| Sodium persulfate | 30 |
| Sodium metasilicate | 14 |
| Ammonium chloride | 5 |
| EDTA | 1 |
| Sodium dioctylsulfosuccinate/sodium benzoate | 1 |
| Calcium stearate | 1 |
| Betulin Oleate in Olive oil (1:1) | 2 |
| Silica | 7 |

40 g of the above anhydrous composition was mixed with 80 g of the following aqueous composition:

Part B:

| | |
|---|---|
| Cetearyl alcohol/ceteareth 30 | 2.85 |
| Stabilizers | 0.06 |
| Sequestering agent | 0.15 |
| Hydrogen peroxide to 200 volumes | 9 |
| Phosphoric acid, qs | pH 2 |
| Distilled water, qsp | 100 |

A bleaching cream ready-for-use was obtained, which applied and left for 45 minutes under a hood, permitted strong and homogeneous bleaching of dark natural hair to be obtained.

### EXAMPLE 16

### Permanent Wave Composition

The following reducing composition was prepared (amounts expressed in grams):

| | |
|---|---|
| Thioglycolic acid | 9.2 |
| Arginine | 15 |
| 20% ammonia NH₃ | 9.3 |
| Ammonium carbonate | 4.5 |
| Cocoylamidopropylbetaine/glycerol monolaurate (25/5) | 1.3 |
| in 30% aqueous solution | |
| Peptizing agent | 0.8 |
| Isostearyl alcohol (Tego Alkanol 66 sold by Goldschmidt) | 12 |
| Betulin Oleate in Olive oil (1:1) | 2.0 |
| Sequestering agent | 0.4 |
| Perfume | 0.4 |
| Demineralized water, qsp | 100 |

This reducing composition was applied to a lock of moist hair wound onto a curler beforehand 9 mm in diameter. After 10 minutes of waiting it was rinsed abundantly with water. The following oxidizing composition was then applied:

Oxidizing Composition:

| Hydrogen peroxide | 8 | volumes |
|---|---|---|
| Tetrasodium pyrophosphate (0.02 g) and sodium | | |
| stannate (0.04 g) | | |
| Sequestering agent: pentasodium pentaacetate | 0.06 | |
| Deionized water, qsp | 100 | g |

After 10 minutes of waiting, the lock was abundantly rinsed again. The hair was then unwound from the curler and dried.

### EXAMPLE 17

### Hair Depilatory

| Raw material | % w/w |
|---|---|
| Phase A | |
| Deionized water | 75.15 |
| Sodium lauryl sulfate | 0.02 |
| Phase B | |
| Ceteryl alcohol (and) ceteth-20 | 6.50 |
| Betulin Oleate | 1.00 |
| Phase C | |
| Calcium thioglycolate | 5.40 |
| Calcium hydroxide | 7.00 |
| Sodium silicate | 3.43 |
| Fragrance | 0.50 |
| Aloe extract | 1.00 |

Heat phase A to 75° C. Melt and mix phase B at 75° C. then add to A while stirring. Cool to room temperature, add C and continue stirring. Stir until homogenous and smooth.

### EXAMPLE 18

### Skin Night Moisturizer

| Raw material | % w/w |
|---|---|
| Phase A | |
| DI water | |
| Methylparaben | |
| Tetrasodium EDTA | |
| Sorbitol (70%) | |
| BHA | |
| Carbomer 934 | |
| Phase B | |
| Triethanolamine | |
| Phase C | |
| Betulin oleate | |
| Mineral oil and lanolin alcohol | |
| Lanolin alcohol | |
| PVP/Eicosene Copolymer | |
| PEG-40 stearate | |
| Propylparaben | |
| Stearic acid | |
| Phase D | |
| Polysorbate 80 | |
| Retinyl palmitate | |
| Phase E | |
| Aloe vera (40%) | |

Combine the ingredients in (A) with the exception of Carbomer and heat to 80-85° C.

### EXAMPLE 19

### Skin Protectant/Sunscreen

| Raw material | % w/w |
|---|---|
| Phase A | |
| Deionized water | 63.35 |
| Carbomer 934 | 0.20 |
| Propylene glycol | 6.25 |
| Cellulose gum | 0.10 |
| Methyl paraben | 0.20 |
| Phase B | |
| Betulin oleate | 2.00 |
| Isoeicosane | 12.00 |
| Isooctahexacontane | 4.00 |
| Steareth-2 | 2.90 |
| Steareth-21 | 2.10 |
| Propylparaben | 0.10 |
| Octyl methoxycinnamate | 6.00 |
| Phase C | |
| Triethanolamine | 0.50 |
| Phase D | |
| Fragrance | 0.30 |

Disperse Phase A and heat to 70-72° C. In a separate vessel combine Phase B and heat to 72-75° C. Mix (A) and (B) and add (C). Mix and cool to 40° C. and add (D).

### EXAMPLE 20

### Skin Moisturizer

| Raw material | % w/w |
|---|---|
| Phase A | |
| Deionized water | 82.40 |
| Hydroxyethyl cellulose | 1.00 |
| Sorbitol | 1.00 |
| Phase B | |
| Stearic acid | 3.00 |
| Glyceryl stearate (and) PEG-100 stearate | 2.50 |
| PEG-75 lanolin oil | 0.50 |
| Cetyl alcohol | 0.50 |
| Betulin oleate | 2.00 |
| Phase C | |
| Collagen amino acids | 5.00 |
| Phase D | |
| Dimethicone | 1.00 |
| Propylene glycol (and) diazolidinyl urea (and) methylparaben | 1.00 |
| (and) propylparaben | |
| Fragrance | 0.10 |

Heat water to 80° C., sprinkle hydroxyethylcellulose into water with constant agitation, add the rest of Phase A, and mix until clear. Melt and mix Phase B ingredients and mix until homogenous. Add slowly Phase B to Phase A. Cool to room temperature. Slowly add collagen aminoacids and mix until smooth. Add D ingredients and mix until uniform.

### EXAMPLE 21

### Antiperspirant

| Raw material | % w/w |
|---|---|
| Phase A | |
| Deionized water | 18.50 |
| Mg—Al silicate, 5% dispersion | 30.00 |
| Phase B | |
| Lanolin alcohol | 0.50 |
| PEG-40 stearate | 2.00 |
| Cetyl alcohol | 1.50 |
| Lanolin | 2.00 |
| Betulin Oleate | 1.00 |
| Phase C | |
| Glycerin | 2.00 |
| Mineral oil | 3.00 |
| Dimethicone | 3.00 |
| Phase D | |
| Aluminum chlorohydrate | 36.50 |
| Fragrance | qs |

Mix A with rapid mixing, heat to 85° C. Mix B at 85° C., add to A while stirring. Then add C. Cool to 40° C. and add D.

### EXAMPLE 22

### Anti-Wrinkle Treatment Cream

| Raw Material | % w/w |
|---|---|
| Phase A | |
| Sodium behenoyl lactylate | 2.00 |
| Cetearyl alcohol | 3.00 |
| Glyceryl stearate | 2.60 |
| Isopropyl palmitate | 6.00 |
| Sunflower seed oil | 6.00 |
| Betulin Oleate | 2.00 |
| Phase B | |
| Deionized water | 70.20 |
| Glycerin | 3.00 |
| Phase C | |
| DMDM hydantoin | 0.20 |
| Sodium lactate (and) lecithin | 6.00 |

Mix A with mixing, heat to 80° C. Heat B to 80° C. Add A to B with vigorous stirring. When homogenous, cool to 35° C. and add C.

### EXAMPLE 23

### Body Wash

| Raw material | % w/w |
|---|---|
| Phase A | |
| Deionized water | qs |
| Sodium cocoamphoacetate | 10.00 |
| Guar hydroxypropyltrimonium chloride | 0.10 |
| (Jaguar 14S) | |
| Citric Acid | qs |
| Phase B | |
| Sodium methyl oleoyl taurate | 1.50 |
| Glycol distearate | 1.50 |
| Ceteareth-20 | 0.30 |
| Betulin Oleate | 1.0 |
| Glycerin | 1.00 |
| Phase C | |
| Sodium laureth sulfate | 45.0 |
| Phase D | |
| Preservative and fragrance | qs |

Combine A by first dispersing the polymer and then adding the rest of ingredients, heat to 75° C. Combine B at the same temperature. Add B to A while mixing. Add C and adjust viscosity with NaCl. Cool to 35° C. and add preservative and fragrance.

1. A personal care composition comprising a mixture of at least one cosmetically acceptable ingredient selected from the group consisting of water, surfactants, emulsifiers, oils, emollients, conditioning agents, protecting agents, fixing agents, polymers, oxidizing agents, reducing agents, dyes, uv absorbers, coloring agents, diluents, solvents, preservatives and mixtures thereof; and an effective amount of a ester or ether of betulin.

2. The personal care composition according to claim 1 wherein said ester or ether of betulin is a compound according to the chemical formula: 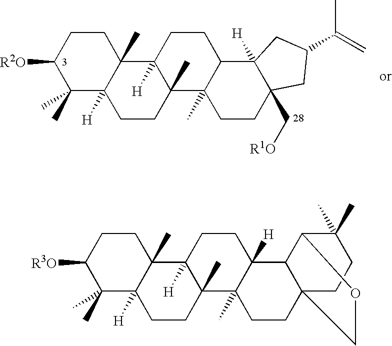
where R¹ and R² are independently H, an optionally substituted acyl group, or forms an optionally substituted ether group with the oxygen to which it is attached, with the proviso that at least one of R¹ and R² is an acyl or a hydrocarbyl group, wherein said optionally substituted acyl group or ether group is linear or branch-chained; and R³ is an optionally substituted acyl group, or forms an optionally substituted ether group with the oxygen to which it is attached, wherein said optionally substituted acyl group or ether group is linear or branch-chained.

3. The personal care composition according to claim 1 comprising a cosmetically acceptable medium in the form of an aqueous dispersion, aqueous emulsion, aqueous solution, oil solution, oil-in-water emulsion, water-in-oil emulsion, oil dispersion, oil-in-water or water-in-oil microemulsion or nanoemulsion.

4. The composition according to claim 1 wherein said ester or ether of betulin is present in the range of about 0.01 to about 50 percent by weight of said composition.

5. The composition according to claim 1 wherein said ester or ether of betulin is a compound according to the formula: 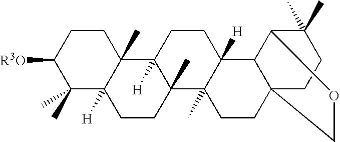
Wherein R³ is an optionally substituted acyl group containing from 1 to about 32 carbon atoms and is present in the composition in the range of about 0.01 to about 50 percent by weight of said composition.

6. The composition according to claim 1 wherein said ester or ether of betulin is a compound according to the formula: 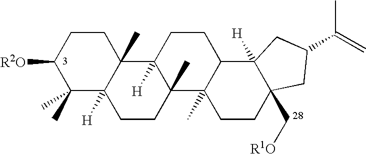
Wherein R¹ and R² are independently H or an optionally substituted acyl group containing from 2 to about 32 carbon atoms with the proviso that both R¹ and R² are not simultaneously H and said compound is present in the composition in the range of about 0.01 to about 50 percent by weight of said composition.

7. The composition according to claim 1 wherein said ester or ether of betulin is a compound according to the formula: 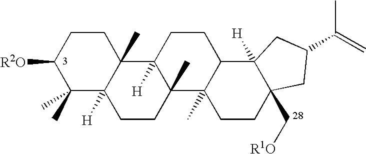
Wherein R¹ and R² are independently H or form an optionally substituted ether group with the oxygen to which they are attached, wherein said ether group contains from 1 to about 32 carbon atoms with the proviso that both R¹ and R² are not simultaneously H and said compound is present in the composition in the range of about 0.01 to about 50 percent by weight of said composition.

8. The composition according to claim 1 wherein said ester or ether of betulin is a compound according to the formula: 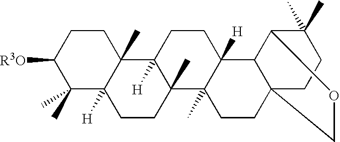
Wherein R³ forms an optionally substituted ether group with the oxygen to which it is attached containing from 1 to about 32 carbon atoms and is present in the composition in the range of about 0.01 to about 50 percent by weight of said composition.

9. The composition according to claim 1 wherein said composition is a hair conditioner, hair shampoo, hair dye, hair bleach or hair perm.

10. The composition according to claim 1 wherein said conditioning agent is selected from the group consisting of synthetic oils, mineral oils, vegetable oils, animal derived oils, fluorinated or perfluorinated oils, natural or synthetic waxes, silicones, cationic polymers, cationic surfactants, proteins and hydrolyzed proteins, ceramide-type compounds, cationic surfactants, fatty amines, fatty acids and derivatives thereof, and mixtures of said conditioning agents.

11. The composition according to claim 1 wherein said conditioning agent is a cationic polymer selected from the group consisting of polymers containing recurring units consisting of primary, secondary, tertiary or quaternary amines, quaternary derivatives of cellulose ether, cationic cyclopolymers, cationic polysaccharides, quaternary polymers of vinyl pyrrolidone and vinyl imidazole, and mixtures thereof.

12. The composition according to claim 1, wherein said conditioning agent is selected from the group consisting of polyalkyl siloxanes, polyaryl siloxanes, polyalkyl aryl siloxanes, silicone gums, resins, organo-modified silicones, and mixtures thereof.

13. The composition according to claim 1, wherein said protecting agent is selected from the group consisting of of sunscreens, photoabsorbers, cationic photoabsorbers, antioxidants, vitamins, and free radical scavengers,

14. The composition according to claim 1 wherein said personal care composition is a skincare product selected from the group consisting of sunscreen cream, sunscreen lotion, suntan cream, body bath, bath gel, skin moisturizer, body lotion, skin-lightening cream, anti-aging cream, slimming gel, anti-cellulite skin conditioning lotion, exfoliating cream, AHA cream, eye cream, shave cream, and after-shave emulsion.

15. The composition according to claim 1 for use in skin and hair treatment for preventing skin conditions caused by parasitic, fungal, or bacterial infections.

16. The composition according to claim 1 for use in skin and hair treatment for preventing skin conditions caused by harmful proteins or toxins.

17. The composition according to claim 1 wherein said composition is selected from the group consisting of floating bath oils, creams, lotions, deodorants, pre-electric shave lotions, after-shave lotions, antiperspirants, deodorants, shampoos, conditioners and rinses and related products, among others, including skin care products such as skin lotions and moisturizers, eye makeups, body shampoos, protective skin formulations, lipsticks, lip glosses, after-bath splashes, presun and sun products, including sunscreens.

18. A compound according to the formula: 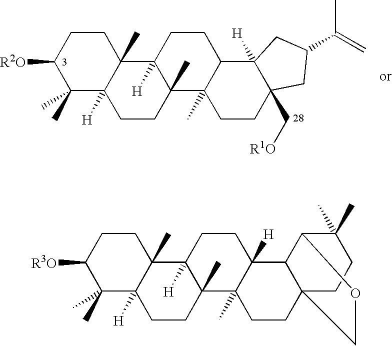
where R¹ and R² are independently H, an optionally substituted acyl group, or forms an optionally substituted ether group with the oxygen to which it is attached, with the proviso that at least one of R¹ and R² is an acyl or a hydrocarbyl group, wherein said optionally substituted acyl group or ether group is linear or branch-chained; and R³ is an optionally substituted acyl group, or forms an optionally substituted ether group with the oxygen to which it is attached, wherein said optionally substituted acyl group or ether group is linear or branch-chained.

19. (canceled)
